(19) United States
(12) Patent Application Publication
(10) Pub. No.: US 2025/0111896 A1
(43) Pub. Date: Apr. 3, 2025

(54) INFORMATION PROCESSING APPARATUS, INFORMATION PROCESSING METHOD, AND PROGRAM

(71) Applicant: SONY GROUP CORPORATION, Tokyo (JP)

(72) Inventor: Satoshi Kawata, Kanagawa (JP)

(73) Assignee: SONY GROUP CORPORATION, Tokyo (JP)

(21) Appl. No.: 18/728,768

(22) PCT Filed: Nov. 29, 2022
(86) PCT No.: PCT/JP2022/043877

§ 371 Date: Jul. 12, 2024

(30) Foreign Application Priority Data
Jan. 21, 2022 (JP) 2022-007704

(51) Int. Cl. G16B 30/00; G16B 15/20; G16B 40/20

(52) U.S. Cl. CPC G16B 30/00; G16B 15/20; G16B 40/20

(57) ABSTRACT

An information processing apparatus according to an embodiment of the present technology includes an acquisition section, an input section, and a generator. The acquisition section acquires protein information related to a protein. Input information that is responsive to an input operation that is performed by a user with respect to the protein information acquired by the acquisition section, is input to the input section. The generator generates sequence information related to an amino acid sequence, on the basis of the protein information acquired by the acquisition section and on the basis of the input information input to the input section. This makes it possible to efficiently generate a desired protein.

### TECHNICAL FIELD

The present technology relates to an information processing apparatus, an information processing method, and a program that are applicable to prediction of amino acid sequences.

### BACKGROUND ART

Patent Literature 1 discloses a prediction system that predicts a protein structure on the basis of amino acid sequences. In the prediction system, processing of aligning amino acid sequences that is called multiple alignment is performed on amino acid sequences, and a protein structure is predicted. This makes it possible to predict a protein structure accurately.

### CITATION LIST

### Patent Literature

Patent Literature 1: United States Patent Application Publication No. 2021/0166779

### DISCLOSURE OF INVENTION

### Technical Problem

Here, there is a need for a technology that makes it possible to generate a desired protein efficiently.

In view of the circumstances described above, it is an object of the present technology to provide an information processing apparatus, an information processing method, and a program that make it possible to generate a desired protein efficiently.

### Solution to Problem

In order to achieve the object described above, an information processing apparatus according to an embodiment of the present technology includes an acquisition section, an input section, and a generator.

The acquisition section acquires protein information related to a protein.

Input information that is responsive to an input operation that is performed by a user with respect to the protein information acquired by the acquisition section, is input to the input section.

The generator generates sequence information related to an amino acid sequence, on the basis of the protein information acquired by the acquisition section and on the basis of the input information input to the input section.

In this information processing apparatus, protein information is acquired, and input information that is responsive to an input operation by a user with respect to the protein information is input. Further, sequence information related to an amino acid sequence is generated on the basis of the protein information and the input information. This makes it possible to efficiently generate a desired protein.

The generator may generate reflection protein information obtained by the input information being reflected in the protein information, and may predict the sequence information corresponding to the reflection protein information.

The generator may predict the sequence information by performing machine learning using the reflection protein information as input.

The protein information may include at least one of a structure of the protein or a function of the protein. In this case, the input operation may include at least one of an editing operation of editing the structure of the protein, or an editing operation of editing the function of the protein.

The function of the protein may include at least one of hydrophilicity of the protein or rigidity of the protein.

The information processing apparatus may further include a protein predictor that predicts, as prediction protein information, the protein information corresponding to the sequence information generated by the generator.

The protein predictor may predict the prediction protein information by performing machine learning using the sequence information as input.

The generator may correct the reflection protein information on the basis of a difference between the reflection protein information and the prediction protein information predicted by the protein predictor.

The information processing apparatus may further include a display controller that controls display of a protein image that corresponds to the protein information acquired by the acquisition section.

The input information may include information that is responsive to the input operation performed with respect to the protein image.

The display controller may control display of a reflection protein image that corresponds to the reflection protein information generated by the generator.

The input information may include information that is responsive to the input operation performed with respect to the reflection protein image.

The display controller may control display of a sequence-information image that corresponds to the sequence information predicted by the generator.

The information processing apparatus may further include a protein predictor that predicts, as prediction protein information, the protein information corresponding to the sequence information predicted by the generator. In this case, the display controller may control display of a difference image that corresponds to a difference between the reflection protein information and the prediction protein information.

The difference image may include an image obtained by the reflection protein image and a prediction protein image that corresponds to the prediction protein information overlapping each other.

The difference image may include an image obtained by the reflection protein image and the prediction protein image overlapping each other, with the difference between the reflection protein information and the prediction protein information being highlighted to be displayed in the included image.

The information processing apparatus may further include a protein predictor that predicts, as prediction protein information, the protein information corresponding to the sequence information generated by the generator. In this case, the display controller may control display of at least one of the protein image, a reflection protein image that corresponds to the reflection protein information generated by the generator, or a prediction protein image that corresponds to the prediction protein information such that the at least one of the protein image, the reflection protein image, or the prediction protein image is displayed in at least one of display formats that respectively correspond to a group-of-points image, a polygon image, a mesh image, a surface image, a slice image, and a three-view diagram.

The protein information may include template information that corresponds to a template for the protein information.

An information processing method according to an embodiment of the present technology is an information processing method that is performed by a computer system, the information processing method including acquiring protein information related to a protein.

Input information that is responsive to an input operation that is performed by a user with respect to the acquired protein information, is input.

Sequence information related to an amino acid sequence is generated on the basis of the acquired protein information and on the basis of the input input information.

A program according to an embodiment of the present technology causes a computer system to perform a process including:

acquiring protein information related to a protein;

inputting input information that is responsive to an input operation that is performed by a user with respect to the acquired protein information; and

generating sequence information related to an amino acid sequence, on the basis of the acquired protein information and on the basis of the input input information.

### BRIEF DESCRIPTION OF DRAWINGS

FIG. 1 schematically illustrates an example of a configuration of a sequence generation system according to an embodiment of the present technology.

FIG. 2 schematically illustrates an example of the configuration of the sequence generation system including a cloud environment.

FIG. 3 schematically illustrates the example of the configuration of the sequence generation system including a cloud environment.

FIG. 4 is a flowchart illustrating an example of processing related to generation of sequence information that is performed by an information processing apparatus.

FIG. 5 is a block diagram illustrating an example of the configuration of the sequence generation system.

FIG. 6 is a flowchart illustrating an example of processing related to prediction of the sequence information.

FIG. 7 schematically illustrates examples of contents displayed on a display section.

FIG. 8 schematically illustrates examples of a machine learning model included in a sequence predictor.

FIG. 9 is a block diagram illustrating an example of the configuration of the sequence generation system.

FIG. 10 is a flowchart illustrating an example of processing related to, for example, generation of a difference image.

FIG. 11 is a flowchart illustrating the example of the processing related to, for example, generation of the difference image.

FIG. 12 schematically illustrates an example of a difference image.

FIG. 13 is a block diagram illustrating an example of the configuration of the sequence generation system.

FIG. 14 is a flowchart illustrating an example of processing related to correction of a reflection stereostructure.

FIG. 15 is a flowchart illustrating the example of the processing related to correction of the reflection stereostructure.

FIG. 16 is a block diagram illustrating an example of a hardware configuration of a computer by which the information processing apparatus can be implemented.

### MODE(S) FOR CARRYING OUT THE INVENTION

Embodiments according to the present technology will now be described below with reference to the drawings.

### [Sequence Generation System]

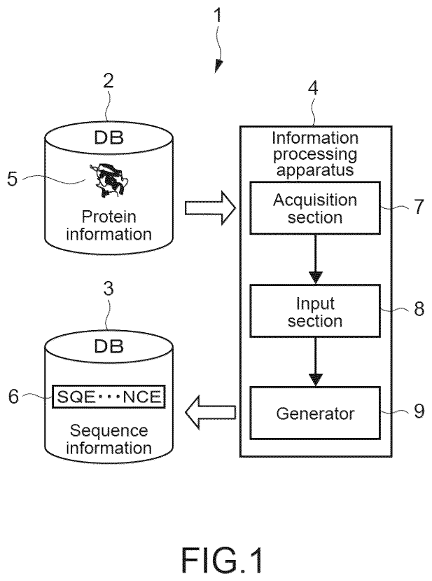

FIG. 1 schematically illustrates an example of a configuration of a sequence generation system according to an embodiment of the present technology.

On the basis of information regarding, for example, a structure of a protein, a sequence generation system 1 can generates and outputs an amino acid sequence included in the protein.

First, a relationship between an amino acid and a protein is described.

Amino acids are linked to form an amino acid sequence. Then, the amino acid sequence is folded to generate a protein.

How an amino acid sequence is folded differs depending on the kind of amino acid sequence from which a protein is generated. Thus, different kinds of proteins are respectively generated from different kinds of amino acid sequences. It can be said that there is a correspondence relationship between an amino acid sequence and a protein, as described above.

The sequence generation system 1 makes it possible to analyze what an amino acid sequence from which a protein is generated is when the protein is given.

The given protein can be specified by a user who uses the sequence generation system 1. Specifically, the user can edit proteins and determine, using editing, a protein to be given.

In other words, when the user thinks that “he/she wants to know what an amino acid sequence from which a certain protein is generated is”, the sequence generation system 1 makes it possible to analyze, by generating the desired protein by editing, the amino acid sequence from which the desired protein is generated.

For example, a structure of an unknown protein can also be input by the user. The sequence generation system 1 also makes it possible to generate and output an amino acid sequence that corresponds to the unknown protein.

As illustrated in FIG. 1, the sequence generation system 1 includes a protein information database (DB) 2, a sequence information DB 3, and an information processing apparatus 4.

The protein information DB 2 is a database that stores therein protein information 5.

The protein information 5 is information related to a protein.

Examples of the protein information 5 include a stereostructure of a protein (a three-dimensional structure and a function that are specific to a protein).

Of course, the protein information 5 may include any other information related to a protein.

The sequence information DB 3 is a database that stores therein sequence information 6.

The sequence information 6 is information related to an amino acid sequence.

Examples of the sequence information 6 include an alphabetic string that represents a sequence.

Typically, the amino acid sequence is a sequence including several tens of amino acid residues to several hundred amino acid residues. If these amino acid residues are expressed using, for example, a rational formula, the formula will be very long.

Thus, an approach of representing the type of amino acid residue using a letter from the alphabet is used in order to represent an amino acid sequence simply. For example, a serine residue is represented by “S”, and a glutamine residue is represented by “Q”. Moreover, all of twenty types of amino acid residues are each represented by a letter from the alphabet.

For example, such an alphabetic string corresponds to the sequence information 6. FIG. 1 schematically illustrates an alphabetic string as the sequence information 6.

Of course, the sequence information 6 may include any other information related to an amino acid sequence.

For example, each of the protein information DB 2 and the sequence information DB 3 is a storage device such as a hard disk drive (HHD) or a solid state drive (SSD). Moreover, any non-transitory computer-readable storage medium may be used.

The information processing apparatus 4 includes hardware, such as a processor including a CPU, a GPU, and a DSP; a memory including a ROM and a RAM; and a storage device including an HDD, that is necessary for a configuration of a computer.

For example, an information processing method according to the present technology is performed by the CPU loading, into the RAM, a program according to the present technology that is recorded in, for example, the ROM in advance and executing the program.

For example, the information processing apparatus 4 can be implemented by any computer such as a personal computer (PC). Of course, hardware such as an FPGA or an ASIC may be used.

In the present embodiment, an acquisition section 7, an input section 8, and a generator 9 are implemented as functional blocks by, for example, the CPU executing a specified program. Of course, dedicated hardware such as an integrated circuit (IC) may be used in order to implement the functional blocks.

The program is installed on the information processing apparatus 4 through, for example, various recording media. Alternatively, the installation of the program may be performed via, for example, the Internet.

The type and the like of a recording medium that records therein a program are not limited, and any computer-readable recording medium may be used. For example, any non-transitory computer-readable recording medium may be used.

The acquisition section 7 acquires the protein information 5.

In the present embodiment, the acquisition section 7 acquires the protein information 5 stored in the protein information DB 2.

Input information that is responsive to an input operation that is performed by a user with respect to the protein information 5 acquired by the acquisition section 7, is input to the input section 8.

For example, the user can edit the protein information 5 by performing an input operation using devices such as a keyboard and a mouse. Input information is input to the input section 8 in response to an input operation being performed by the user.

The generator 9 generates the sequence information 6 on the basis of the protein information 5 acquired by the acquisition section 7 and on the basis of the input information input to the input section 8.

The sequence information 6 generated by the generator 9 is output to the sequence information DB 3.

Further, the generator 9 controls display of the sequence information 6 that is performed by a display device (for example, a display of a PC).

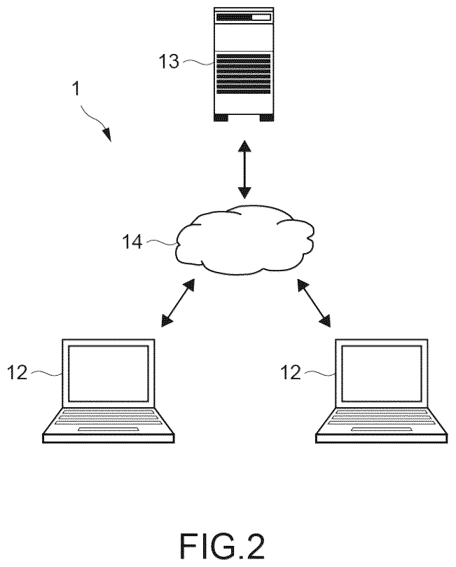

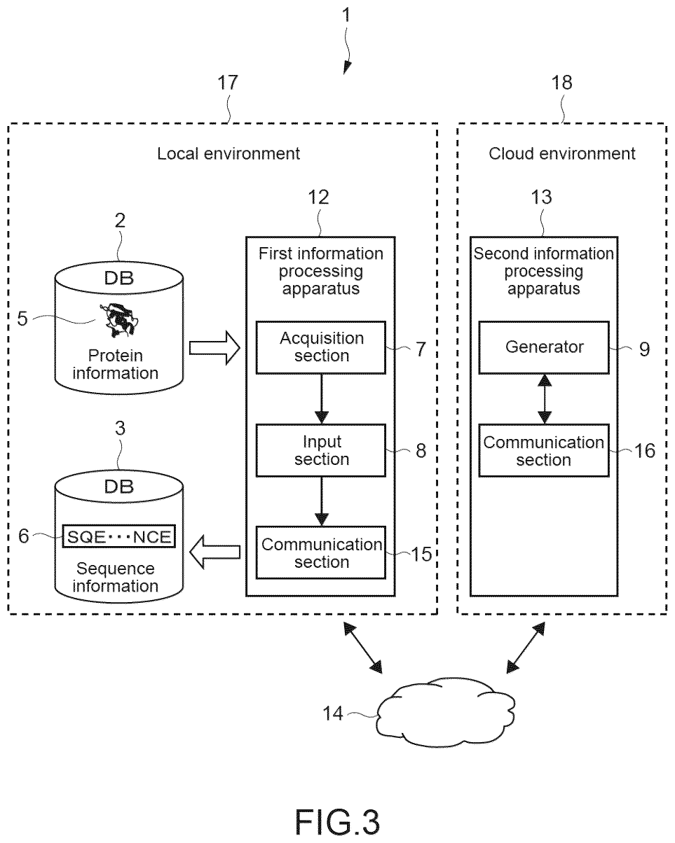

FIGS. 2 and 3 schematically illustrate an example of the configuration of the sequence generation system 1 including a cloud environment.

In this example, the sequence generation system 1 includes two first information processing apparatuses 12 and a second information processing apparatus 13.

The terminal and the apparatus are connected through a network 14 to be capable of communicating with each other. The network 14 is built by, for example, the Internet or a wide area communication network. Moreover, for example, any wide area network (WAN) or any local area network (LAN) may be used, and a protocol used to build the network 14 is not limited.

Further, as illustrated in FIG. 3, the sequence generation system 1 includes the protein information DB 2 and the sequence information DB 3. Note that illustrations of the protein information DB 2 and the sequence information DB 3 are omitted in FIG. 2.

The first information processing apparatus 12 includes the acquisition section 7 and input section 8 illustrated in FIG. 1, and a communication section 15.

The communication section 15 is a module used to perform, for example, network communication or near field communication with another device. For example, a wireless LAN module such as Wi-Fi, or a communication module such as Bluetooth (registered trademark) is provided as the communication section 15.

The communication section 15 transmits, to the network 14, the protein information 5 acquired by the acquisition section 7 and the input information input to the input section 8. Further, the communication section 15 receives the sequence information 6 transmitted by the second information processing apparatus 13 through the network 14.

The second information processing apparatus 13 includes the generator 9 illustrated in FIG. 1, and a communication section 16.

The communication section 16 receives, through the network 14, the protein information 5 and input information being transmitted by the first information processing apparatus 12. Further, the second information processing apparatus 13 transmits, to the network 14, the sequence information 6 generated by the generator 9.

In this example, for example, an apparatus such as a PC that can be operated by a user is used as the first information processing apparatus 12. Input information is input by the user performing an input operation through an input device in order to edit the protein information 5. The input information is transmitted to the second information processing apparatus 13 together with the protein information 5.

The second information processing apparatus 13 is, for example, a server apparatus, and the sequence information 6 is generated on the basis of the received protein information 5 and input information. Further, the sequence information 6 is transmitted to the first information processing apparatus 12, and, for example, the sequence information 6 is displayed on a screen of the first information processing apparatus 12, and the sequence information 6 is output to the sequence information DB 3.

As described above, the sequence generation system 1 may include an environment (a local environment 17) on the side of a user and an environment (a cloud environment 18) situated at a location distant from the user.

In the configuration example illustrated in FIGS. 2 and 3, functions according to the present technology that are included in the information processing apparatus 4 are implemented by the first information processing apparatus 12 and the second information processing apparatus 13 working cooperatively.

In other words, in the configuration example illustrated in FIGS. 2 and 3, an information processing apparatus according to the present technology is implemented and an information processing method according to the present technology is performed by two computers working cooperatively, the two computers being connected through the network 14 to be capable of communicating with each other.

In this example, two first information processing apparatuses 12 that can be operated by a user in the local environment 17 are arranged, as illustrated in FIG. 2. A plurality of first information processing apparatuses 12 may be arranged in the local environment 17, as described above, and the sequence generation system 1 may be usable by a plurality of users. Of course, the number of first information processing apparatuses 12 arranged in the local environment 1 is not limited, and at least three first information processing apparatuses 12 may be arranged.

Further, a configuration in which the first information processing apparatus 12 and the second information processing apparatus 13 are connected through, for example, a cable to be capable of communicating with each other may also be adopted.

Furthermore, a configuration in which the protein information DB 2 and the sequence information DB 3 are in the cloud environment 18 may also be adopted.

Moreover, a specific configuration of the sequence generation system 1 is not limited.

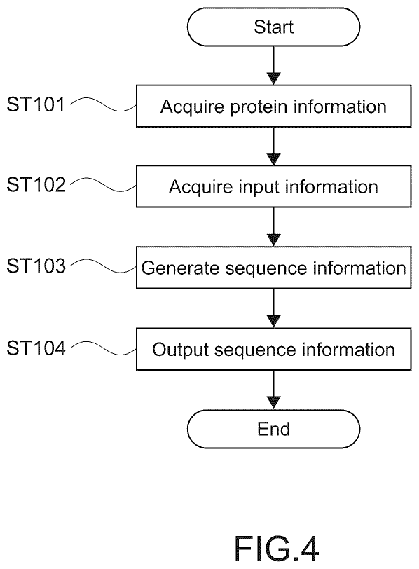

FIG. 4 is a flowchart illustrating an example of processing related to generation of the sequence information 6 that is performed by the information processing apparatus 4. In the configuration example illustrated in FIGS. 2 and 3, the processing example illustrated in FIG. 4 is performed by the first information processing apparatus 12 and the second information processing apparatus 13 working cooperatively.

The acquisition section 7 acquires the protein information 5 (Step 101).

Specifically, the acquisition section 7 acquires the protein information 5 stored in the protein information DB 2.

The input section 8 acquires input information (Step 102).

For example, the input information is acquired by the input section 8 in response to a user performing an input operation in order to edit the protein information 5.

Note that the acquisition of the input information that is performed by the input section 8 is included in the input of the input information to the input section 8.

The generator 9 generates the sequence information 6 (Step 103).

Specifically, first, the generator 9 acquires the protein information 5 from the acquisition section 7, and acquires the input information from the input section 8. Further, the sequence information 6 is generated on the basis of the protein information 5 and the input information.

In the present embodiment, the generator 9 generates the sequence information 6 by processing being performed using a machine learning algorithm. A method for generating the sequence information 6 will be described in detail later.

The sequence information 6 generated by the generator 9 is output (Step 104).

In the present embodiment, the generator 9 outputs the sequence information 6 to the sequence information DB 3. This results in the sequence information 6 being stored in the sequence information DB 3.

Further, the sequence information 6 is displayed on a display device such as a display of a PC. The display of the sequence information 6 on the display device is included in the output of the sequence information 6.

Note that, when the sequence generation system 1 including the cloud environment 18 is adopted, processing related to communication between the first information processing apparatus 12 and the second information processing apparatus 13 is performed just before Step 103 (the generation of sequence information) and just before Step 104 (the output of sequence information).

### First Embodiment

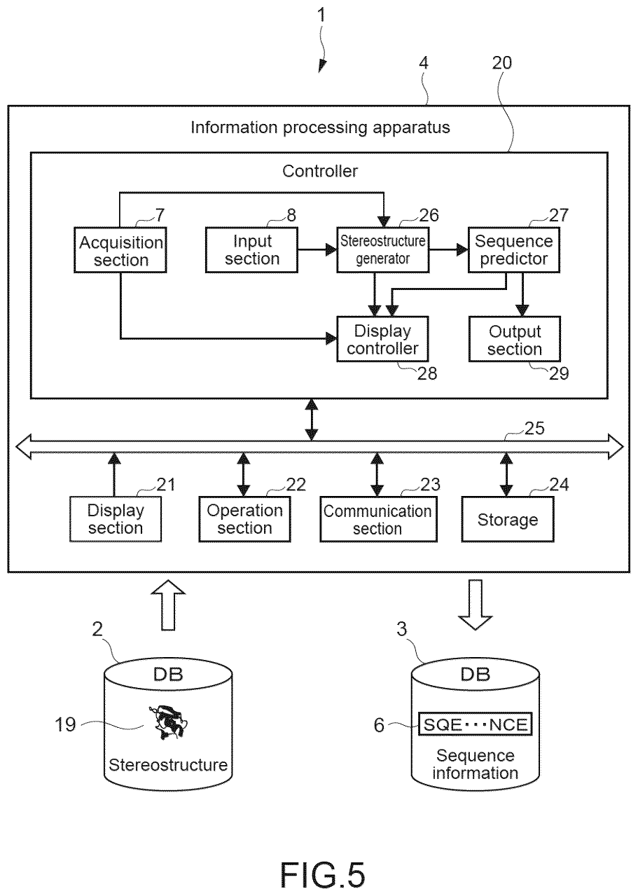

A more detailed embodiment of the sequence generation system 1 according to the present technology is described as a first embodiment with reference to FIGS. 5 to 8.

FIG. 5 is a block diagram illustrating an example of the configuration of the sequence generation system 1.

The sequence generation system 1 includes the protein information DB 2, the sequence information DB 3, and the information processing apparatus 4.

The protein information DB 2 stores therein a stereostructure 19 as the protein information 5.

The stereostructure 19 is information that includes a three-dimensional structure and a function that are specific to a protein.

The stereostructure 19 includes at least one of a protein structure or a protein function.

The protein structure corresponds to information related to a protein structure. Examples of the protein structure include information including coordinates columns of three-dimensional coordinates of, for example, atoms, molecules, bonds, and functional groups that are included in a protein. The coordinates columns of the three-dimensional coordinates may be referred to as volume data.

Of course, specifically what kind of information the protein structure is, is not limited, and any information related to a protein structure may be included.

The protein function corresponds to information related to a protein function.

In the present embodiment, the protein function includes at least one of hydrophilicity of a protein or rigidity of the protein.

A certain protein has a structure of which a portion has a local hydrophilicity. Further, a certain protein has a structure of which a portion has a local rigidity (the property of not being easily folded).

Examples of the protein function include function labels that represent such hydrophilicity and such rigidity.

The function labels correspond to a numerical value that represents a range of three-dimensional coordinates of a portion that has the hydrophilicity or the rigidity, and a numerical value that represents a degree of hydrophilicity or a degree of rigidity.

Conversely, the function label may include a numerical value that represents, for example, a range of three-dimensional coordinates of a portion that has the hydrophobicity or the non-rigidity.

Further, when a protein locally has a Y-shaped structure, a function of capturing a virus using arms of the Y-shape may appear. The protein function may include a function label that represents such an immune function.

Moreover, specifically what kind of information the protein function is, is not limited, and any information related to a protein function may be included.

Note that contents of the protein information 5 are not limited to the protein structure or the protein function. For example, any information related to a protein, such as an image related to the protein, may be stored in the protein information DB 2.

Further, in the present embodiment, template information that corresponds to a template for the protein information 5 is stored in the protein information DB 2 as the protein information 5.

The template information is the protein information 5, which corresponds to an initial value and is provided in order for a user to perform editing.

For example, a user selects, from a plurality of pieces of template information, a piece of template information close to the protein information 5 the user wants to generate. This makes it possible to reduce amounts of time and labor that are necessary to perform editing, compared to when editing is started in a state in which no information is provided.

For example, template information is generated by an administrator of the sequence generation system 1 in advance and stored in the protein information DB 2.

Alternatively, data of proteins made publicly available in the database of the Worldwide Protein Data Bank (wwPDB) may be used as the template information. In this case, the template information is generated using a data format such as a PDB format, a PDBML format, or an mmCIF format.

Moreover, specific contents of the template information are not limited.

For example, the sequence information DB 3 stores therein, as the sequence information 6, an alphabetic string that represents a sequence of amino acid residues.

Of course, specific contents of the sequence information 6 are not limited. For example, any information related to an amino acid sequence, such as information regarding amino acid sequences represented by, for example, a structural formula and a rational formula, may be stored in the sequence information DB 3.

The information processing apparatus 4 includes a controller 20, a display section 21, an operation section 22, a communication section 23, and a storage 24.

The controller 20, the display section 21, the operation section 22, the communication section 23, and the storage 24 are connected to each other through a bus 25. The respective blocks may be connected to each other using, for example, a communication network or an unstandardized unique communication approach instead of using the bus 25.

The display section 21 is a display device using, for example, liquid crystal or electroluminescence (EL), and, for example, various images and various graphical user interfaces (GUIs) are displayed on the display section 21.

Examples of the operation section 22 include a keyboard, a pointing device, a touchscreen, and other operation apparatuses. When the operation section 22 includes a touchscreen, the touchscreen may be integrated with the display section 21.

In the present embodiment, input information is generated in response to an input operation being performed by a user through the operation section 22.

The communication section 23 is a module used to perform, for example, network communication or near field communication with another device.

When, for example, the sequence generation system 1 includes the cloud environment 18, the communication section 23 communicates with the network 14.

The storage 24 is a storage device such as a nonvolatile memory, and, for example, an HDD or an SSD is used. Moreover, any non-transitory computer-readable storage medium may be used as the storage 24.

The storage 24 stores therein a control program used to control an operation of the overall information processing apparatus 4. A method for installing the control program on the information processing apparatus 4 is not limited.

For example, the installation may be performed through various recording media, or the installation of the program may be performed through, for example, the Internet.

Further, the storage 24 may store therein the stereostructure 19 or the sequence information 6.

The controller 20 includes hardware, such as a processor including a CPU, a GPU, and a DSP; a memory including a ROM and a RAM; and a storage device including an HDD, that is necessary for a configuration of a computer. For example, the information processing method according to the present technology is performed by the CPU loading, into the RAM, a program according to the present technology that is recorded in, for example, the ROM in advance and executing the program.

For example, a programmable logic device (PLD) such as a field programmable gate array (FPGA), or another device such as an application specific integrated circuit (ASIC) may be used as the controller 20.

In the present embodiment, the acquisition section 7, the input section 8, a stereostructure generator 26, a sequence predictor 27, a display controller 28, and an output section 29 are implemented as functional blocks by the CPU of the controller 20 executing the program according to the present technology (such as an application program).

Then, an information processing method according to the present embodiment is performed by these functional blocks. Note that, in order to implement each functional block, dedicated hardware such as an integrated circuit (IC) may be used as appropriate.

The acquisition section 7 acquires the protein information 5.

In the present embodiment, the acquisition section 7 acquires the stereostructure 19 corresponding to template information from the protein information DB 2.

Further, the acquisition section 7 outputs the stereostructure 19 to the display controller 28.

The input section 8 acquires input information.

The input information is information that is responsive to an input operation that is performed by a user with respect to the stereostructure 19 acquired by the acquisition section 7.

For example, a user can perform editing work on a screen while checking an image related to the stereostructure 19 displayed on the display section 21. Specifically, for example, the user can perform various editing work such as changing an arrangement of an atom by performing a dragging operation of dragging an image of the atom using a mouse included in the operation section 22.

In this case, the “dragging operation” corresponds to the input operation. Further, for example “new coordinates of an atom” correspond to the input information. The “new coordinates of an atom” corresponding to the input information are determined according to, for example, the track of the “dragging operation” corresponding to the input operation.

Specific contents of the input information are not limited, and any information that is responsive to the input operation is included.

The stereostructure generator 26 generates a reflection stereostructure obtained by input information being reflected in the stereostructure 19.

For example, it is assumed that the stereostructure 19 acquired by the acquisition section 7 includes three-dimensional coordinates indicating that “coordinates of an atom A are represented by X=10, Y=20, and Z=30” and the input information is information indicating that “new coordinates of the atom A are represented by X=20, Y=10, and Z=40”. In this case, the reflection stereostructure is information that includes three-dimensional coordinates indicating that “the coordinates of the atom A are represented by X=20, Y=10, and Z=40”.

Note that, of course, examples of the stereostructure 19 and the reflection stereostructure may also include information including coordinates and the types of a plurality of atoms; coordinates and the types of molecules, bonds, and functional groups; and function labels.

Further, the stereostructure generator 26 outputs the reflection stereostructure to the sequence predictor 27 and the display controller 28.

The reflection stereostructure corresponds to an embodiment of reflection protein information according to the present technology.

The sequence predictor 27 predicts the sequence information 6 corresponding to the reflection stereostructure.

In the present embodiment, the sequence information 6 is predicted by a method using a machine learning algorithm.

Note that the prediction of the sequence information 6 that is performed by the sequence predictor 27 is included in the generation of the sequence information 6.

The sequence information 6 predicted by the sequence predictor 27 is output to the display controller 28 and the output section 29.

The display controller 28 controls display of an image corresponding to the stereostructure 19 acquired by the acquisition section 7. Further, the display controller 28 controls display of an image corresponding to the reflection stereostructure generated by the stereostructure generator 26, and display of an image corresponding to the sequence information 6 predicted by the sequence predictor 27.

The output section 29 outputs, in the form of a file, the sequence information 6 predicted by the sequence predictor 27.

Specifically, the output section 29 outputs the sequence information 6 to the sequence information DB 3. Alternatively, the sequence information 6 may be output to the storage 24 or a specified storage medium.

Further, the sequence information 6 may be output to a storage medium in the cloud environment 18 through the communication section 23.

Moreover, a specific output destination to which the output section 29 outputs the sequence information 6 is not limited.

A format of, for example, a text file, a FASTA file, or a csv file is used as a file format of the sequence information 6.

Without being limited thereto, any format such as an image format may be adopted.

In the present embodiment, an embodiment of a generator according to the present technology is implemented by the stereostructure generator 26, the sequence predictor 27, and the output section 29.

The configurations of the controller 20, the display section 21, the operation section 22, the communication section 23, and the storage 24 that are described with reference to FIG. 5 are merely examples, and specific configurations thereof are not limited.

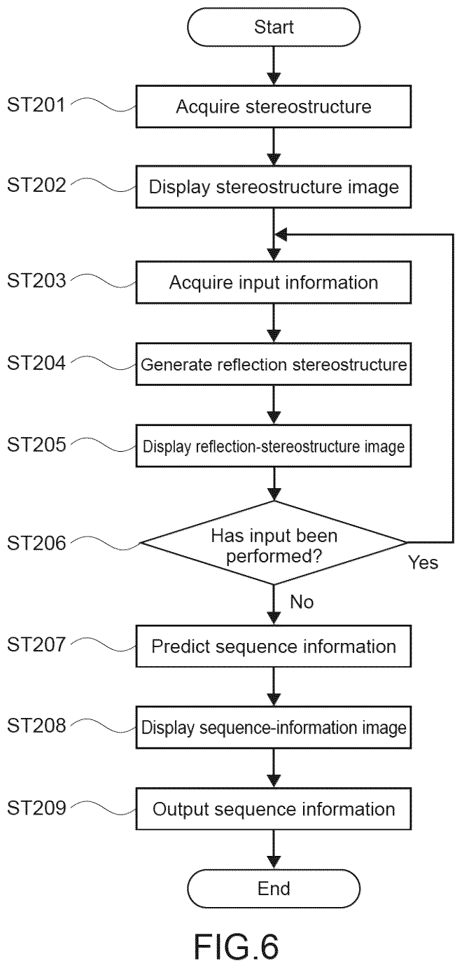

FIG. 6 is a flowchart illustrating an example of processing related to prediction of the sequence information 6.

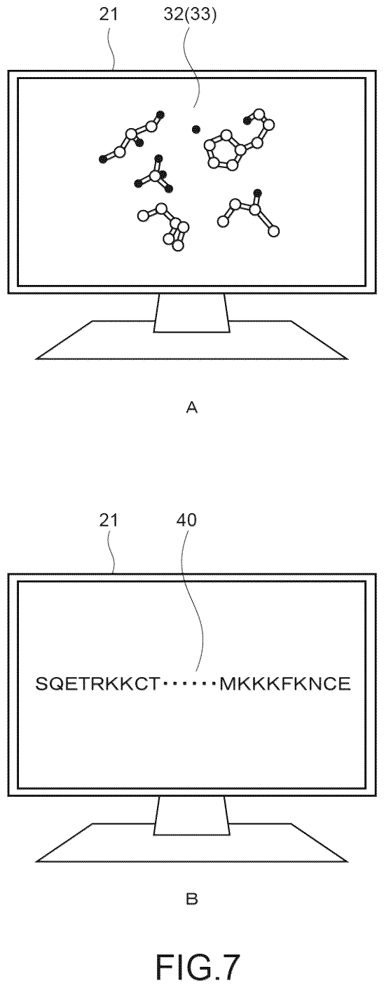

FIG. 7 schematically illustrates examples of contents displayed on the display section 21.

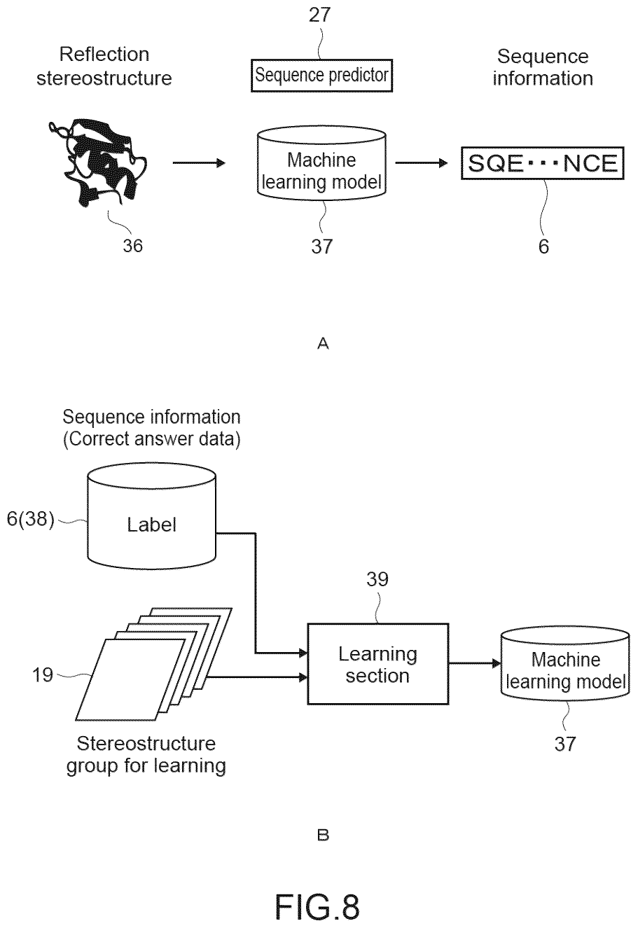

FIG. 8 schematically illustrates examples of a machine learning model included in the sequence predictor 27.

First, the acquisition section 7 acquires the stereostructure 19 (Step 201).

### [Display of Stereostructure Image]

A stereostructure image is displayed on the display section 21 (Step 202).

In the present embodiment, the display controller 28 controls display of a stereostructure image corresponding to the stereostructure 19 acquired by the acquisition section 7.

Specifically, first, the display controller 28 acquires the stereostructure 19 from the acquisition section 7. Further, a stereostructure image corresponding to the stereostructure 19 is generated, and display of the stereostructure image that is performed on the display section 21 is controlled.

A of FIG. 7 schematically illustrates a stereostructure image 32 that is being displayed on the display section 21.

In the present embodiment, the display controller 28 controls display of the stereostructure image 32 such that the stereostructure image 32 is displayed in at least one of display formats that respectively correspond to a group-of-points image, a polygon image, a mesh image, a surface image, a slice image, and a three-view diagram.

The group-of-points image is an image obtained by representing data using a set of points. For example, atoms included in a protein are respectively represented by points to be displayed in the form of a group-of-points image.

Specifically, a position of a point in a group-of-points image is calculated on the basis of three-dimensional coordinates of an atom included in the stereostructure 19, and the group-of-points image is generated.

Of course, a specific method for generating a stereostructure image such as a group-of-points image is not limited.

Not only an atom but also, for example, a molecule, a functional group, a function label, a principal chain of a protein, and a side chain of the protein may be represented by points to be displayed in the form of a group-of-points image.

Alternatively, points may be displayed in different colors according to the type of atom or the type of function label.

Moreover, specific contents displayed in the form of a group-of-points image are not limited.

Note that the group of points may be referred to as a point cloud.

The polygon image is an image obtained by representing data using a polygon. For example, a local shape of a protein is represented by a triangle or a quadrilateral.

The mesh image is an image obtained by representing data using a plurality of polygons. For example, a shape of a protein is represented by a shape obtained by putting triangles and quadrilaterals together. It can also be said that the mesh image is a collection of polygon images.

The surface image is an image obtained by representing data using a smooth curve. For example, a shape of a protein is represented by a smooth curve.

The slice image is an image obtained by representing a cross section of a protein. For example, a cross section at a specified position in a group-of-points image is displayed in the form of a slice image. Alternatively, a cross section of a polygon image, a mesh image, or a surface image may be displayed.

The three-view diagram includes images obtained by representing shapes of a protein as viewed from three directions. Examples of the three-view diagram include diagrams of a protein as viewed from any directions, such as a front view, a top view, a bottom view, a right side view, a left side view, and a rear view, with a specified surface of the protein being the front.

Display of the stereostructure image 32 in one of the display formats enables a user to intuitively recognize, for example, a protein structure.

Further, the slice image enables a user to easily recognize an internal structure of a protein (a structure invisible from the outside).

Note that, for example, the display format, a position of a cross section in a slice image, and directions used for a three-view diagram can be changed by a user as appropriate using, for example, a setting button.

Moreover, a specific display format in which the stereostructure image 32 is displayed is not limited.

The stereostructure image 32 corresponds to an embodiment of a protein image according to the present technology.

### [Input Operation]

The input section 8 acquires input information (Step 203).

In the present embodiment, the input information includes information that is responsive to an input operation that is performed with respect to the stereostructure image 32.

In other words, a user can perform an input operation with respect to the stereostructure image 32 while checking the stereostructure image 32 displayed on the display section 21. The stereostructure 19 is edited, as described above.

When, for example, editing of “changing an arrangement of an atom” is performed, a “dragging operation of dragging a point representing an atom in the stereostructure image 32” is performed as an input operation.

This operation is an input operation performed with respect to the stereostructure image 32.

Further, in the present embodiment, the input operation includes at least one of an editing operation of editing a protein structure or an editing operation of editing a protein function.

For example, the “changing an arrangement of an atom” corresponds to the editing a protein structure, and the “dragging operation of dragging a point representing an atom in the stereostructure image 32” corresponding to the “changing an arrangement of an atom” corresponds to the editing operation of editing a protein structure.

Other variations of the edition of a protein structure and the editing operation of editing a protein structure are described.

For example, editing such as not only a change in an arrangement of an atom but also changes in a new arrangement of the atom, a deletion of the atom, a selection of the atom, and the type of the atom (such as α carbon, β carbon, oxygen, and nitrogen), can also be performed.

Such editing is performed by, for example, a clicking operation of clicking a point representing an atom in the stereostructure image 32, or a dragging operation of dragging the point.

In this case, the input section 8 acquires, as the input information, information such as “deleting an atom A”, and “a new type of atom A is carbon”.

Alternatively, a molecule, a functional group, a principal chain of a protein, and a side chain of the protein may be editable in a similar manner. In this case, editing such as deformation of, for example, a molecule may be performable.

Further, atoms and others may be collectively arrangeable in a desired region.

In other words, not only a method for precisely arranging atoms and others at respective points, but also, for example, a method including specifying a desired region using a dragging operation and collectively arranging all of atoms and others in the entirety of the desired region, may be adopted.

Likewise, all of atoms and others in a region may be selectable, movable, or deletable.

Further, how atoms are linked may be editable.

For example, two atoms are specified by a clicking operation, and a selection screen used to select the type of bond is displayed with a right-clicking. Further, a desired type (such as hydrogen bond) is selected using, for example, a check box.

Furthermore, only a skeletal structure (a rough shape) of a protein may be specified by a user, and a detailed arrangement of atoms and others may be automatically determined according to the specified skeletal structure.

Other variations of the edition of a protein function and the editing operation of editing a protein function are described.

For example, function labels respectively representing functions of “hydrophilicity”, “hydrophobicity”, “rigidity”, and “non-rigidity” can be locally added.

For example, a user selects a desired region using a dragging operation, and then selects, using, for example, a check box, a function label the user wants to add.

In this case, the input information is, for example, information indicating that “a new function of a function label A is hydrophilicity and a range of coordinates is represented by X=10 to 20, Y=10 to 30, and Z=20 to 40”.

When a function label is added, an arrangement of atoms and others is automatically determined on the basis of, for example, the added function label.

When, for example, a function label “hydrophilicity” is added to a certain region, the arrangement of atoms and others in the region is automatically determined such that a protein has a “hydrophilicity” function in the region.

This also makes it possible to add a function when a user wants a protein to have a desired function but is not sure how to arrange atoms and others.

Note that template information acquired by the acquisition section 7 may be information in which only a position of an atom is determined and the type of, for example, the atom is not determined. In this case, for example, a user himself/herself specifies the type of, for example, the atom by editing.

Of course, the template information, such as data from the Worldwide Protein Data Bank, in which a position and a structure of, for example, the atom are determined, may be acquired by the acquisition section 7.

Moreover, specific contents of, for example, the edition of a protein structure, the edition of a protein function, the input operation, and the input information are not limited.

Further, in order to perform an editing operation, any graphical user interfaces (GUIs) such as various windows, various buttons, various check boxes, various tabs, and various entry fields may be arranged.

Note that the input operation is not limited to the input operation performed with respect to images.

For example, editing may be performable by an input operation, such as input of a text or sound recognition, that is other than the input operation performed with respect to images.

The stereostructure generator 26 generates a reflection stereostructure (Step 204).

Specifically, the stereostructure generator 26 acquires the stereostructure 19 from the acquisition section 7, and acquires the input information from the input section 8. Further, the reflection stereostructure is generated on the basis of the acquired stereostructure 19 and input information.

When, for example, the type of an atom A included in the stereostructure 19 is oxygen and when the input information indicates that “a new type of the atom A is carbon”, the reflection stereostructure corresponds to information obtained by replacing oxygen with carbon with respect to the atom A in the stereostructure 19.

As described above, the input information is reflected in the stereostructure 19 to generate the reflection stereostructure.

A reflection-stereostructure image is displayed on the display section 21 (Step 205).

In the present embodiment, the display controller 28 controls display of a reflection-stereostructure image corresponding to the reflection stereostructure generated by the stereostructure generator 26.

Specifically, first, the display controller 28 acquires a reflection stereostructure from the stereostructure generator 26. Then, a reflection-stereostructure image corresponding to the reflection stereostructure is generated, and display of the reflection-stereostructure image that is performed on the display section 21 is controlled.

As in the case of the stereostructure image 32, a reflection-stereostructure image 33 is generated on the basis of three-dimensional coordinates of, for example, an atom included in the reflection stereostructure.

For example, the example illustrated in A of FIG. 7 can also be considered to be an example of displaying the reflection-stereostructure image 33.

When, for example, the reflection-stereostructure image 33 is newly displayed, the stereostructure image 32 originally displayed in Step 202 is deleted. The reflection-stereostructure image 33 may be displayed in the same display format as the display format used for the originally displayed stereostructure image 32, or may be displayed in a display format different from the display format used for the originally displayed stereostructure image 32.

Alternatively, both the stereostructure image 32 and the reflection-stereostructure image 33 may overlap to be displayed in the same display format, without the stereostructure image 32 being deleted. This enables a user to easily recognize how contents edited by the user are reflected.

The reflection-stereostructure image 33 corresponds to an embodiment of a reflection protein image according to the present technology.

It is determined whether input to the operation section 22 has been performed (Step 206).

In the present embodiment, a user can further edit the reflection stereostructure. In this case, the user performs, for example, a clicking operation with respect to the reflection-stereostructure image 33.

An affirmative determination is made when an input operation associated with editing has been performed. Determination is performed on the basis of, for example, whether the input section 8 has acquired an input operation.

When it has been determined that input to the operation section 22 has been performed (Yes in Step 206), the input section 8 acquires input information again (Step 203).

The input information includes information that is responsive to an input operation performed with respect to the reflection-stereostructure image 33.

Then, on the basis of the reflection stereostructure and the input information, the stereostructure generator 26 generates a new reflection stereostructure (Step 204).

Further, a new reflection-stereostructure image 33 is displayed on the display section 21 (Step 205).

When it has been determined that input to the operation section 22 has not been performed (No in Step 206), the sequence predictor 27 predicts the sequence information 6 (Step 207).

When, for example, input has not been performed for a certain period of time, it has been determined that input has not been performed. Alternatively, sequence prediction processing illustrated in Step 207 may start being performed when, for example, an input termination button or a sequence prediction button is pressed.

### [Prediction of Sequence Information]

In the present embodiment, the sequence predictor 27 predicts the sequence information 6 corresponding to a reflection stereostructure.

Specifically, first, the sequence predictor 27 acquires a reflection stereostructure from the stereostructure generator 26. Then, the sequence information 6 is predicted on the basis of the acquired reflection stereostructure.

Further, in the present embodiment, the sequence predictor 27 performs machine learning using a reflection stereostructure as input to predict the sequence information 6.

A of FIG. 8 schematically illustrates an example of predicting the sequence information 6 using a learning model using a reflection stereostructure as input.

As illustrated in A of FIG. 8, a reflection stereostructure 36 is input to a machine learning model 37 that has performed learning using machine learning performed to estimate the sequence information 6. Then, the machine learning model 37 predicts the sequence information 6.

This makes it possible to predict the sequence information 6 with a high degree of accuracy.

B of FIG. 8 is a schematic diagram used to describe learning performed by the machine learning model 37 using training data.

In the present embodiment, the stereostructure 19 is used as data for learning. Data obtained by the sequence information 6 (a training label 38) being associated with the data for learning, is used as training data.

Thus, the machine learning model 37 is a prediction model that has performed machine learning using the stereostructure 19 and the sequence information 6 as training data.

As illustrated in B of FIG. 8, a learning section 39 uses training data to perform learning on the basis of a machine learning algorithm. Accordingly, the machine learning model 37 is generated.

In the present embodiment, first, a graphical model or a distance map is generated on the basis of the data for learning (the stereostructure 19). The graphical model or the distance map is generated by, for example, the sequence predictor 27.

Then, the graphical model or the distance map, and the sequence information 6 (the training label 38) are input to the learning section 39 to perform learning. Thus, it can also be said that a pair of the graphical model generated from the stereostructure 19 and the sequence information 6 (the training label 38), or a pair of the distance map generated from the stereostructure 19 and the sequence information 6 is used as training data.

The graphical model is a graph that represents a dependence in probability. Specifically, the graphical model includes a plurality of nodes and a plurality of edges. The nodes are connected to each other using an edge, where it is often the case that the node is schematically represented by a circle and the edge is schematically represented by a line that connects the nodes.

For example, a length of an edge connecting two nodes is determined according to the probability related to the two nodes. The length of the edge is relatively small if the probability is relatively high, and the length of the edge is relatively large if the probability is relatively low.

In the present embodiment, the graphical model is generated on the assumption that the node corresponds to an atom and the edge corresponds to the probability that the atoms are linked to each other.

For example, when the probability that an atom A and an atom B are linked to each other is high, a node representing the atom A and a node representing the atom B are connected to each other using a short edge.

On the other hand, when the probability that the atom A and the atom B are linked to each other is low, the nodes are connected to each other using a long edge.

Note that it is known that the probability that atoms are linked to each other is dependent on a distance between the atoms.

For example, the probability that atoms are linked to each other is high when a distance between the atoms are small. On the other hand, the probability that the atoms are linked to each other is low when the distance between the atoms are large.

In other words, a graphical model may be generated on the assumption that a distance between atoms corresponds to an edge.

In this case, nodes are connected to each other using a long edge when a distance between atoms is large. This also means that the probability that the atoms are linked to each other is low.

Conversely, nodes are connected to each other using a short edge when a distance between atoms is small. This also means that the probability that the atoms are linked to each other is high.

Further, atoms may be connected using an edge only when a distance between the atoms is less than a specified threshold (for example, 10 angstroms). A pair of atoms between which a distance is less than a threshold (considered to be in contact with each other) may be referred to as a pair of contact atoms.

Further, function labels may be embedded in a node and an edge. In other words, a feature amount of node and a feature amount of edge may be generated on the basis of function labels.

Moreover, a specific method for generating a graphical model is not limited.

The distance map is a map on which a distance between atoms is shown.

For example, a two-dimensional square map is used as a distance map.

For example, each atom included in a protein is assigned a number. Further, for example, a distance between an atom with “No. 30” and an atom with “No. 50” is represented by lightness in monochrome at a position represented by “X=30 and Y=50” on the distance map.

For example, when a distance between atoms is small, a color at a corresponding position is close to white. Conversely, when the distance is large, the color at a corresponding position is close to black.

Moreover, the distance may be represented by, for example, lightness, saturation, or hue in color.

Further, a contact map may be generated as the distance map.

The contact map is a two-dimensional square map that is similar to the distance map. The contact map is included in the distance map.

When a distance between atoms is less than a specified threshold, a color at a corresponding position is white on the contact map. Conversely, when the distance is greater than the specified threshold, the color at a corresponding position is black on the contact map.

As described above, a distance between atoms is represented by “0 or 1” on the contact map.

Learning is performed on the basis of the generated graphical model or distance map.

Upon learning, the sequence information 6 represented using, for example, one-hot encoding is used as the training label 38.

The one-hot encoding is a display format used to display data including a dummy variable (0).

Specifically, an amino acid residue is represented by 20 digits using one-hot encoding. For example, “serine(S)”, which is the sixteenth amino acid, is represented by a numerical string “00000000000000010000”, which includes “1” only at the sixteenth place and “0” at each of the other places.

Likewise, when, for example, an amino acid sequence including five amino acids is represented using one-hot encoding, the amino acid sequence is represented by a numerical string of 100 digits.

Note that, in the description above, “serine(S)” is determined to be the sixteenth amino acid by defining the order of amino acid using the alphabetical order used to note amino acids. However, of course, a method for determining the order is not limited.

A specific algorithm used to perform learning using a graphical model or a distance map and using the sequence information 6 is not limited, and learning may be performed using, for example, a known approach.

Note that a graphical model, a distance map, or a contact map may be included in the stereostructure 19 and stored in the protein information DB 2.

A sequence-information image is displayed on the display section 21 (Step 208).

In the present embodiment, the display controller 28 controls display of a sequence-information image corresponding to the sequence information 6 predicted by the sequence predictor 27.

Specifically, first, the display controller 28 acquires the sequence information 6 from the sequence predictor 27. Further, a sequence-information image corresponding to the sequence information 6 is generated, and display of the sequence-information image that is performed on the display section 21 is controlled.

B of FIG. 7 schematically illustrates a sequence-information image 40 that is being displayed on the display section 21.

In this example, an alphabetic string that represents an amino acid sequence is displayed as the sequence-information image 40.

Without being limited thereto, any image, such as an image of a structural formula or a rational formula of an amino acid sequence, that corresponds to the sequence information 6 may be displayed.

The output section 29 outputs the sequence information 6 (Step 208).

Specifically, first, the output section 29 acquires the sequence information 6 from the sequence predictor 27. Further, the sequence information is output to, for example, the sequence information DB 3, the storage 24, and other storage media.

This enables a user to easily manage the predicted sequence information 6.

A processing order of displaying (Step 208) the sequence-information image 40 and outputting (Step 209) the sequence information 6 in the processing illustrated in FIG. 6 may be reversed. In other words, the sequence-information image 40 may be displayed after the sequence information 6 is output.

Moreover, specific contents of processing related to prediction of the sequence information 6 are not limited.

In the sequence generation system 1 according to the present embodiment, the stereostructure 19 is acquired, and input information that is responsive to an input operation performed by a user with respect to the stereostructure 19 is input, as described above. Further, the sequence information 6 related to an amino acid sequence is generated on the basis of the stereostructure 19 and the input information. This makes it possible to efficiently generate a desired protein.

A protein is formed by several tens of amino acids to several hundred amino acids being linked by peptide bonds, and is folded into a specific three-dimensional structure in a cell to be provided with a function.

For example, a certain type of antibody protein has a structure formed to catch a virus and an antigen, and this results in the certain type of antibody protein acting in a state of being immune.

The protein structure is directly associated with a protein function. Thus, understanding of a protein structure is a very important research task.

However, a relationship between a one-dimensional amino acid sequence and a three-dimensional protein structure has not been sufficiently understood in the past.

Thus, there is a need to repeat trial and error in culturing a microorganism and in performing an experimental analysis in order to generate a protein having a desired structure and a desired function upon synthesizing an organic compound. This results in a great deal of time and effort and huge costs.

In recent years, a method and an apparatus that are used to predict a shape of a stereostructure using a neural network that uses an amino acid sequence as input, have been proposed.

Such a structure prediction technology has greatly evolved in recent years. For example, such a structure prediction technology is also used to analyze a structure of Covid-19, and contributes toward a rapid development of vaccines.

Further, modeling of a protein structure that is performed using a graphical model by use of an encoder and a decoder, has also been proposed.

On the other hand, there is still an issue of what kinds of amino acid sequences are to be generated in order to obtain a desired stereostructure. With respect to such an issue, an approach of predicting an amino acid sequence using a graph neural network that uses a stereostructure as input, has also been proposed.

There is a need for a further new approach, as described above, that is used to solve an issue about a low throughput (efficiency) in synthesizing an organic compound.

The sequence generation system according to the present technology provides protein designing software to a user. The user generates and edits protein information, and this enables the user to interactively design a desired protein.

This makes it possible to greatly improve a throughput in a cycle for culturing and analysis upon synthesizing an organic compound and performing drug discovery.

Further, in the present embodiment, the reflection stereostructure 36 obtained by input information being reflected in the stereostructure 19 is generated, and the sequence information 6 corresponding to the reflection stereostructure 36 is predicted.

This results in editing contents being reflected with a high degree of accuracy, and in predicting the sequence information 6 accurately.

Furthermore, in the present embodiment, a structure and a function of a protein can be edited. Further, editing can be performed with respect to hydrophilicity and rigidity of a protein, which is editing of the function.

This enables a user to perform editing with a high degree of freedom. Furthermore, editing can be performed while imagining a function of a protein to be obtained.

Further, the stereostructure image 32 and the reflection-stereostructure image 33 are displayed on the display section 21.

This enables a user to perform editing while checking how his/her own editing operation is reflected.

Further, the sequence-information image 40 is displayed on the display section 21.

This enables a user to easily recognize what predicted sequence information is.

Further, an input operation for editing can be performed with respect to the stereostructure image 32 and the reflection-stereostructure image 33.

This enables a user to perform editing by performing a simple, easy, and intuitive operation.

### Second Embodiment

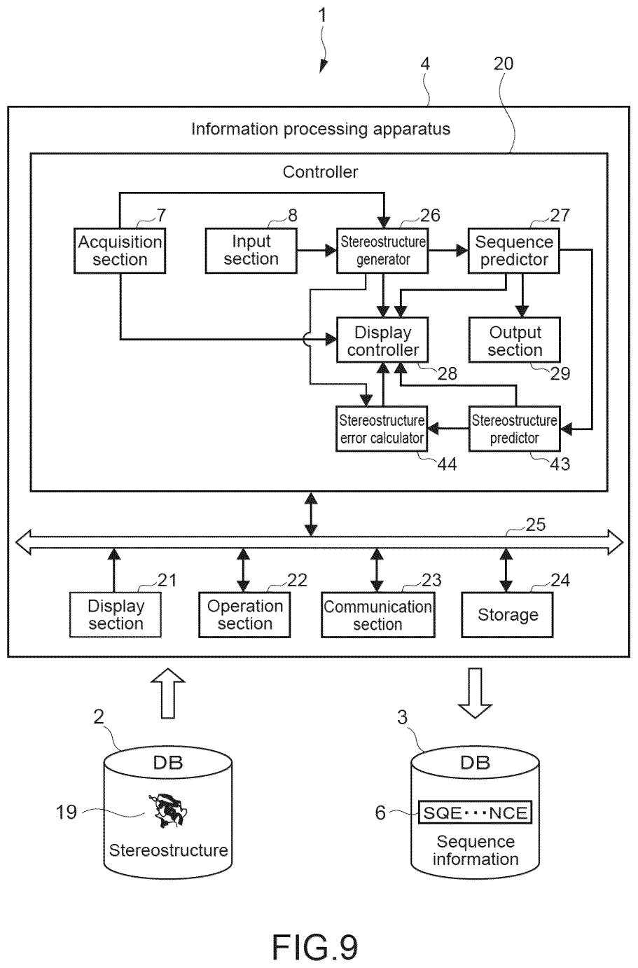

A more detailed embodiment of the sequence generation system 1 according to the present technology is described as a second embodiment with reference to FIGS. 9 to 11.

In the following description, descriptions of a configuration and an operation that are similar to those of the sequence generation system 1 described in the embodiment above are omitted or simplified.

In the present embodiment, the stereostructure 19 is further predicted on the basis of the sequence information 6 predicted by the sequence predictor 27.

FIG. 9 is a block diagram illustrating an example of the configuration of the sequence generation system 1.

In the present embodiment, a stereostructure predictor 43 and a stereostructure error calculator 44 are further implemented as functional blocks by the CPU of the controller 20 executing the program according to the present technology.

The stereostructure predictor 43 predicts, as a prediction stereostructure, the stereostructure 19 corresponding to the sequence information 6 predicted by the sequence predictor 27.

Specifically, first, the stereostructure predictor 43 acquires the sequence information 6 from the sequence predictor 27. Further, a prediction stereostructure is predicted on the basis of the acquired sequence information 6.

When a certain protein is generated from an amino acid sequence represented by the sequence information 6, information related to the certain protein is predicted as a prediction stereostructure.

In other words, in the present embodiment, the stereostructure 19 is information related to a protein A, the sequence information 6 is information related to an amino acid sequence from which the protein A is generated, and the prediction stereostructure is “information related to a protein that is generated from the amino acid sequence from which the protein A is generated”.

In other words, the stereostructure 19 and the prediction stereostructure are pieces of information analogous to each other in principle.

On the other hand, the sequence information 6 and the prediction stereostructure are generated by prediction processing being performed. Thus, an error may occur in the course of prediction. Therefore, there is a possibility that the stereostructure 19 and the prediction stereostructure will not be exactly identical to each other and thus an error will occur.

The prediction stereostructure predicted by the stereostructure predictor 43 is output to the display controller 28 and the stereostructure error calculator 44.

The stereostructure predictor 43 corresponds to an embodiment of a protein predictor according to the present technology.

The prediction stereostructure corresponds to an embodiment of prediction protein information according to the present technology.

The stereostructure error calculator 44 calculates a difference between the reflection stereostructure 36 and the prediction stereostructure predicted by the stereostructure predictor 43.

Note that the difference can also be referred to as an error. In the following description, a difference in information between the reflection stereostructure 36 and the prediction stereostructure may be referred to as a difference or an error. Which of the words is selectively used has no particular significance.

The difference calculated by the stereostructure error calculator 44 is output to the display controller 28.

The stereostructure generator 26, the sequence predictor 27, the output section 29, and the stereostructure predictor 43 correspond to an embodiment of the generator according to the present technology.

Further, in the present embodiment, the display controller 28 controls display of a difference image that corresponds to the difference between the reflection stereostructure 36 and the prediction stereostructure.

Specifically, first, the display controller 28 acquires the reflection stereostructure 36 from the stereostructure generator 26, and acquires the prediction stereostructure from the stereostructure predictor 43. Further, a difference image is generated on the basis of the acquired reflection stereostructure 36 and prediction stereostructure, and display of the difference image that is performed on the display section 21 is controlled.

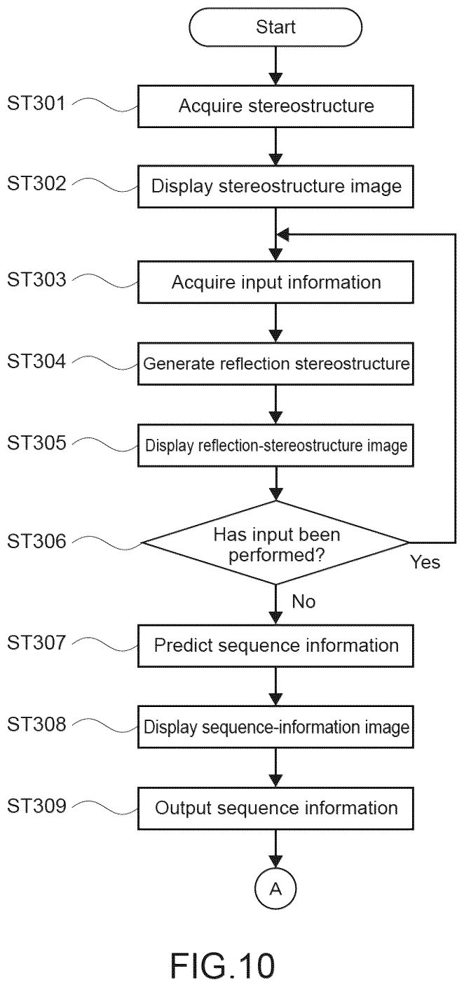

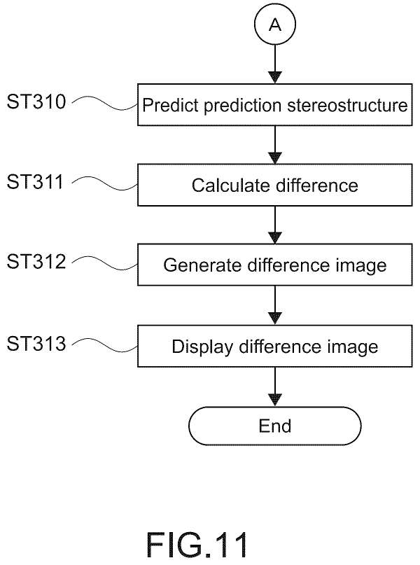

FIGS. 10 and 11 are flowcharts illustrating an example of processing related to, for example, generation of a difference image.

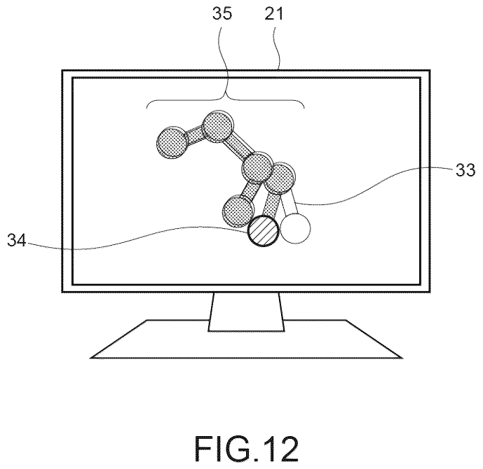

FIG. 12 schematically illustrates an example of a difference image.

Processing similar to the processing performed in Steps 201 to 209 illustrated in FIG. 6 is performed in Steps 301 to 309 illustrated in FIG. 10.

A prediction stereostructure is predicted by the stereostructure predictor 43 (Step 310).

In the present embodiment, the stereostructure predictor 43 predicts the prediction stereostructure by performing machine learning using the sequence information 6 as input.

This makes it possible to predict a prediction stereostructure with a high degree of accuracy.

Training data obtained by the sequence information 6 (data for learning) and the stereostructure 19 (a training label) being associated with each other, is used to perform learning.

A specific algorithm used to perform learning is not limited, and learning may be performed using, for example, a known approach.

The stereostructure error calculator 44 calculates a difference (Step 311).

For example, the stereostructure error calculator 44 calculates, as a difference, a shift between sets of coordinates of an atom that is included in the reflection stereostructure 36 and the prediction stereostructure in common.

When coordinates of an atom A in the reflection stereostructure 36 are represented by “X=20, Y=10, and Z=40” and coordinates of the atom A in the prediction stereostructure are represented by “X=22, Y=13, and Z=39”, the difference to be calculated corresponds to information that indicates “X=2, Y=3, and Z=−1”.

Alternatively, the difference may be calculated using average root-mean-square deviation (RMSD) or mean absolute error (MAE), which is used as an indicator.

Further, a difference between the types of, for example, atoms situated at respective positions identical to each other may be calculated as the difference.

When, for example, an atom situated at a certain position in the reflection stereostructure 36 is carbon and an atom situated at a corresponding position in the prediction stereostructure is oxygen, a difference to be calculated corresponds to information that indicates “the types of atoms are different”. Alternatively, information that includes the types of respective atoms, that is, information that indicates “the types of atoms are carbon and oxygen” may be calculated.

Moreover, any information that indicates a difference in information between the reflection stereostructure 36 and a prediction stereostructure, such as a shift between positions to which a function is added, a difference between the types of functions, a difference between positions of bonds, or a difference between the types of bonds, may be calculated as the difference.

The display controller 28 generates a difference image (Step 312).

In the present embodiment, the display controller 28 generates, as the difference image, an image obtained by the reflection-stereostructure image 33 and a prediction-stereostructure image overlapping each other, the prediction-stereostructure image corresponding to the prediction stereostructure.

Specifically, first, the display controller 28 acquires a prediction stereostructure from the stereostructure predictor 43. Further, a prediction-stereostructure image is generated on the basis of the acquired prediction stereostructure. Furthermore, on the basis of the reflection-stereostructure image 33 generated in Step 305 and on the basis of the prediction-stereostructure image, a difference image obtained by the reflection-stereostructure image 33 and the prediction-stereostructure image overlapping each other.

FIG. 12 illustrates the reflection-stereostructure image 33 in white. Further, the prediction-stereostructure image 34 is hatched in the figure. Furthermore, an image obtained by these images overlapping each other is a difference image 35.

On the basis of the prediction stereostructure, the prediction-stereostructure image 34 is generated by a method similar to the method for generating the stereostructure image 32.

The prediction-stereostructure image 34 corresponds to an embodiment of a prediction protein image according to the present technology.

When, for example, the reflection stereostructure 36 and the prediction stereostructure are identical to each other, the reflection-stereostructure image 33 and the prediction-stereostructure image 34 are images identical to each other. In this case, the difference image 35 is an image obtained by images identical to each other overlapping each other. Thus, the difference image 35 is an image in which a single stereostructure 19 seems to appear.

On the other hand, when an error occurs between the reflection stereostructure 36 and the prediction stereostructure, the difference image 35 is an image in which two stereostructures 19 shifted from each other seem to appear.

FIG. 12 illustrates an example in which an error occurs between the reflection stereostructure 36 and the prediction stereostructure, with the reflection-stereostructure image 33 and the prediction-stereostructure image 34 being shifted from each other.

Further, in the present embodiment, the display controller 28 generates, as the difference image 35, an image obtained by the reflection-stereostructure image 33 and the prediction-stereostructure image 34 overlapping each other, with a difference between the reflection stereostructure 36 and the prediction stereostructure being highlighted to be displayed in the generated image.

Specifically, the display controller 28 acquires a difference from the stereostructure error calculator 44. Further, on the basis of the reflection-stereostructure image 33, the prediction-stereostructure image 34, and the difference, the difference image 35 obtained by the two images overlapping each other is generated, with the difference being highlighted to be displayed in the difference image 35.

For example, a position at which there is a relatively large difference is highlighted to be displayed.

Specifically, when a difference in coordinate values at a certain position is greater than a specified threshold, a corresponding position in an image obtained by two images overlapping each other is displayed in a different color.

In the example illustrate in FIG. 12, atoms are shifted from each other in a lower right portion. Thus, a corresponding portion is highlighted to be displayed.

Further, not only a position at which there is a relatively large difference in coordinate values, but also a position at which there is a relatively large difference in, for example, the type of, for example, atom, a position of a function label, or the type of function label may be highlighted to be displayed.

Conversely, a position at which there is a relatively small difference may be highlighted to be displayed.

Note that a specific highlighting-and-displaying method is not limited. For example, the highlighting and displaying may be performed using, for example, blinking or gradation.

Of course, an image that does not include a highlighted and displayed portion may be generated as the difference image 35.

Further, any difference image 35 other than the image obtained by the reflection-stereostructure image 33 and the prediction-stereostructure image 34 overlapping each other may be generated. For example, an image obtained by two images being just simply arranged side by side may be generated.

The difference image 35 is displayed on the display section 21 (Step 313).

Specifically, the display controller 28 controls display of the difference image 35 that is performed on the display section 21.

This enables a user to evaluate appropriateness of predicted sequence information 6.

Further, the user can intuitively recognize an error between the reflection stereostructure 36 generated by the user performing editing, and a predicted prediction stereostructure.

For example, a position, in the difference image 35, at which there is a large shift is checked, and a corresponding position in the reflection stereostructure 36 is edited in order to correct for an error. As described above, the stereostructure 19 can be edited efficiently, and a throughput in synthesizing an organic compound can be improved.

Note that an error may be presented to a user by a difference being displayed using not only an image but also a concrete numerical value.

Moreover, a specific method for presenting an error to a user is not limited.

### Third Embodiment

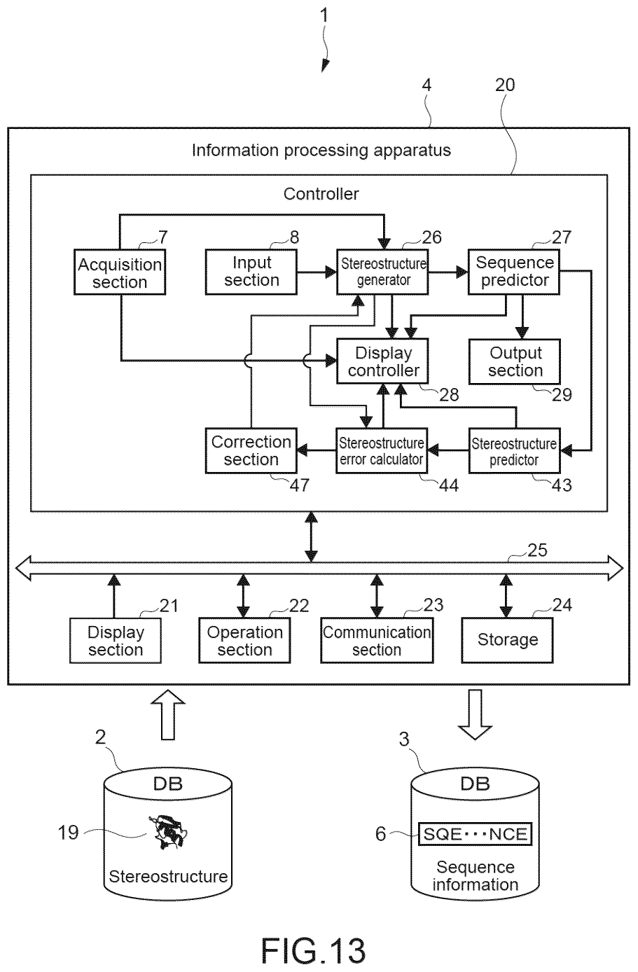

A more detailed embodiment of the sequence generation system 1 according to the present technology is described as a third embodiment with reference to FIGS. 13 to 15.

In the present embodiment, the reflection stereostructure 36 is automatically corrected on the side of the sequence generation system 1 on the basis of a difference.

FIG. 13 is a block diagram illustrating an example of the configuration of the sequence generation system 1.

In the present embodiment, a correction section 47 is further implemented as a functional block by the CPU of the controller 20 executing the program according to the present technology.

The correction section 47 generates correction information on the basis of a difference calculated by the stereostructure error calculator 44.

An embodiment of the generator according to the present technology is implemented by the stereostructure generator 26, the sequence predictor 27, the output section 29, the stereostructure predictor 43, and the correction section 47.

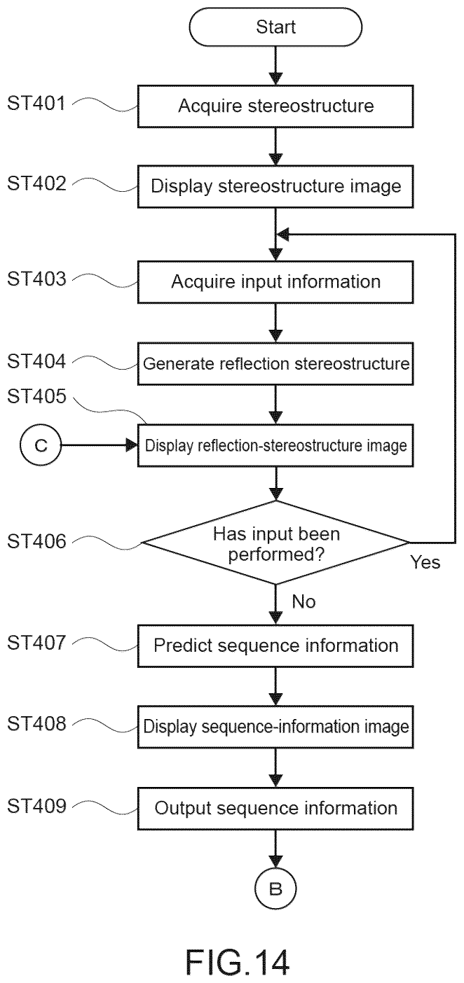

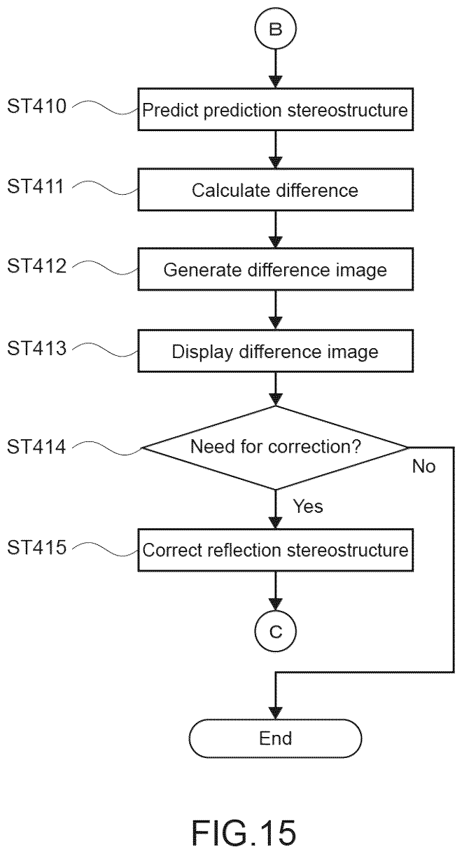

FIGS. 14 and 15 are flowcharts illustrating an example of processing related to correction of the reflection stereostructure 36.

Processing similar to the processing performed in Steps 301 to 313 illustrated in FIGS. 10 and 11 is performed in Steps 401 to 413.

It is determined whether there is a need to correct the reflection stereostructure 36 (Step 414).

When, for example, a difference (an error) is greater than a specified threshold, it is determined that there is a need for the correction. Alternatively, when a user presses a correction button, it may be determined that there is a need for the correction.

For example, the determination is performed by the correction section 47.

When it has been determined that there is a need for the correction (Yes in Step 414), the reflection stereostructure 36 is corrected (Step 415).

In the present embodiment, the correction section 47 and the stereostructure generator 26 correct the reflection stereostructure 36 on the basis of a difference between the reflection stereostructure 36 and the prediction stereostructure predicted by the stereostructure predictor 43.

Specifically, first, the correction section 47 acquires a difference from the stereostructure error calculator 44. Then, correction information is generated on the basis of the acquired difference.

When, for example, coordinates of an atom A in the reflection stereostructure 36 are represented by “X=20, Y=10, and Z=40” and coordinates of the atom A in the prediction stereostructure are represented by “X=22, Y=13, and Z=39”, the difference corresponds to information that indicates “X=2, Y=3, and Z=−1”.

In this case, the correction section 47 generates, as correction information, information that indicates “adding ‘X=+2, Y=+3, and Z=−1’ to coordinate values of the atom A”.

The correction information generated by the correction section 47 is output to the stereostructure generator 26.

On the basis of the correction information acquired from the correction section 47, the stereostructure generator 26 generates a correction stereostructure obtained by the reflection stereostructure 36 being corrected.

For example, on the basis of the correction information, “X=+2, Y=+3, and Z=−1” is added to “X=20, Y=10, and Z=40”, which corresponds to the coordinates of the atom A in the reflection stereostructure 36. Consequently, a correction stereostructure with which the coordinates of the atom A correspond to “X=22, Y=13, and Z=39” is generated.

The generated correction stereostructure includes information identical to information included in the prediction stereostructure.

In other words, in this example, processing of correcting the reflection stereostructure 36 itself into the prediction stereostructure, is performed.

Of course, specific contents of the correction are not limited. For example, a “structure obtained by averaging” the reflection stereostructure 36 and the prediction stereostructure may be generated as a prediction stereostructure. In this case, for example, calculation is performed to obtain an average of sets of coordinate values for each atom.

Alternatively, any information such as the type of atom, positions of a molecule and others, the types of a molecule and others, a position of a bond, the type of bond, a position of a function label, and the type of function label may be corrected.

Further, when the reflection stereostructure 36 and the prediction stereostructure are identical to each other (when there is no error therebetween), correction does not necessarily have to be performed.

After the stereostructure generator 26 generates the correction stereostructure, the reflection-stereostructure image 33 is displayed on the display section 21 again (Step 405).

The reflection-stereostructure image 33 is generated on the basis of the correction stereostructure. In other words, the reflection-stereostructure image 33 in which correction has been reflected is displayed.

A user can further edit the correction stereostructure by performing an operation with respect to the reflection-stereostructure image 33.

When it has been determined that there is no need for the correction (No in Step 414), the processing is terminated.

In the present embodiment, the reflection stereostructure 36 is automatically corrected on the side of the sequence generation system 1 on the basis of a difference (an error). This results in omitting correction of the reflection stereostructure 36 that is performed by a user. This makes it possible to efficiently design a protein.

### Other Embodiments

The present technology is not limited to the embodiments described above, and can achieve various other embodiments.

In the second embodiment or the third embodiment, the sequence predictor 27 may perform learning using, as training data, the prediction stereostructure predicted by the stereostructure predictor 43 and the sequence information 6 predicted by the sequence predictor 27.

This makes it possible to predict sequence prediction with a high degree of accuracy.

In this case, especially in the third embodiment, correction processing is performed repeatedly multiple times and the sequence information 6 and the prediction stereostructure are predicted every time the processing is performed. The sequence predictor 27 may perform learning using the predicted sequence information 6 and prediction stereostructure every time the processing is performed. This makes it possible to further improve the accuracy in prediction performed by the sequence predictor 27.

A portion of or all of the functions of the protein information DB 2 or sequence information DB 3 illustrated in FIG. 1 may be included in the information processing apparatus 4. Alternatively, a portable information processing apparatus 4 may be used, and a portion of or all of the functions of the protein information DB 2 or the sequence information DB 3 may be included in the portable information processing apparatus 4.

The sequence generation system 1 may be implemented by a plurality of computers or a single computer.

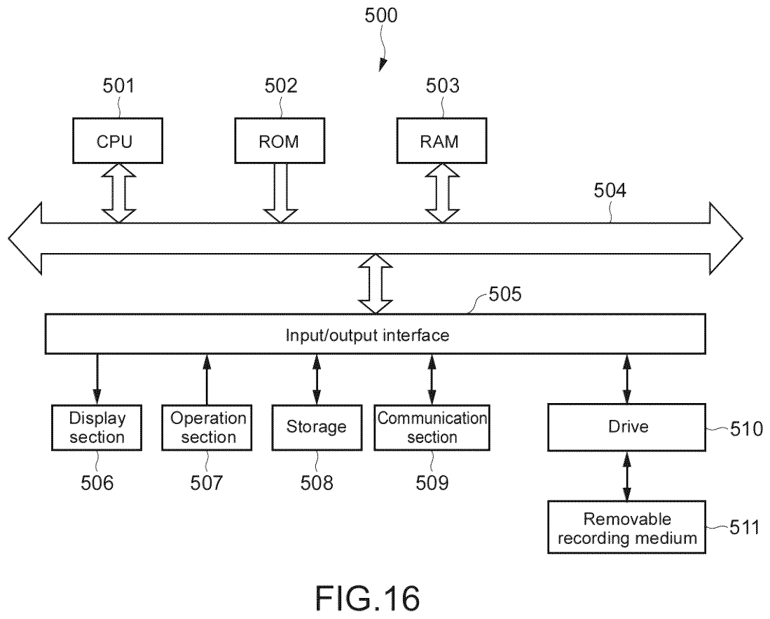

FIG. 16 is a block diagram illustrating an example of a hardware configuration of a computer 500 by which the information processing apparatus 4 can be implemented.

The computer 500 includes a CPU 501, a ROM 502, a RAM 503, an input/output interface 505, and a bus 504 through which these components are connected to each other. A display section 506, an operation section 507, a storage 508, a communication section 509, a drive 510, and the like are connected to the input/output interface 505.

The display section 506 is a display device using, for example, liquid crystal or EL. Examples of the operation section 507 include a keyboard, a pointing device, a touchscreen, and other operation apparatuses. When the operation section 507 includes a touchscreen, the touchscreen may be integrated with the display section 506.

The storage 508 is a nonvolatile storage device, and examples of the storage 508 include an HDD, a flash memory, and other solid-state memories. The drive 510 is a device that can drive a removable recording medium 511 such as an optical recording medium or a magnetic recording tape.

The communication section 509 is a modem, a router, or another communication apparatus that can be connected to, for example, a LAN or a WAN and is used to communicate with another device. The communication section 509 may perform communication wirelessly or by wire. The communication section 509 is often used in a state of being separate from the computer 500.

Information processing performed by the computer 500 having the hardware configuration described above is performed by software stored in, for example, the storage 508 or the ROM 502, and hardware resources of the computer 500 working cooperatively. Specifically, the information processing method according to the present technology is performed by loading, into the RAM 503, a program included in the software and stored in the ROM 502 or the like and executing the program.

For example, the program is installed on the computer 500 through the removable recording medium 511. Alternatively, the program may be installed on the computer 500 through, for example, a global network. Moreover, any non-transitory storage medium that is readable by the computer 500 may be used.

The information processing method according to the present technology may be executed and the sequence generation system and the information processing apparatus according to the present technology may be implemented by a plurality of computers working cooperatively, the plurality of computers being a plurality of computers connected through, for example, a network to be capable of communicating with each other.

In other words, the information processing method according to the present technology can be executed not only in a computer system that includes a single computer, but also in a computer system in which a plurality of computers operates cooperatively.

Note that, in the present disclosure, the system refers to a set of components (such as apparatuses and modules (parts)) and it does not matter whether all of the components are in a single housing. Thus, a plurality of apparatuses accommodated in separate housings and connected to each other through a network, and a single apparatus in which a plurality of modules is accommodated in a single housing are both the system.

The execution of the information processing method according to the present technology by the computer system includes, for example, both the case in which the acquisition of protein information, the input of input information, the generation and the correction of reflection protein information, the prediction and the output of sequence information, the prediction of prediction protein information, the calculation of a difference, the generation of correction information, the display of, for example, a protein image, and the like are executed by a single computer; and the case in which the respective processes are executed by different computers. Further, the execution of the respective processes by a specified computer includes causing another computer to execute a portion of or all of the processes and acquiring a result of it.

In other words, the information processing method according to the present technology is also applicable to a configuration of cloud computing in which a single function is shared and cooperatively processed by a plurality of apparatuses through a network.

The sequence generation system, the information processing apparatus, the contents displayed by the display section, the respective processing flows, and the like described with reference to the respective figures are merely embodiments, and any modifications may be made thereto without departing from the spirit of the present technology. In other words, for example, any other configurations or algorithms for purpose of practicing the present technology may be adopted.

When wording such as “substantially” is used in the present disclosure, such wording is merely used to facilitate the understanding of the description, and whether the wording such as “substantially” is used has no particular significance.

In other words, in the present disclosure, expressions, such as “center”, “middle”, “uniform”, “equal”, “similar”, “orthogonal”, “parallel”, “symmetric”, “extend”, “axial direction”, “columnar”, “cylindrical”, “ring-shaped”, “annular”, and “average” that define, for example, a shape, a size, a positional relationship, and a state respectively include, in concept, expressions such as “substantially the center/substantial center”, “substantially the middle/substantially middle”, “substantially uniform”, “substantially equal”, “substantially similar”, “substantially orthogonal”, “substantially parallel”, “substantially symmetric”, “substantially extend”, “substantially axial direction”, “substantially columnar”, “substantially cylindrical”, “substantially ring-shaped”, “substantially annular”, and “substantially average”.

For example, the expressions such as “center”, “middle”, “uniform”, “equal”, “similar”, “orthogonal”, “parallel”, “symmetric”, “extend”, “axial direction”, “columnar”, “cylindrical”, “ring-shaped”, “annular”, and “average” also respectively include states within specified ranges (such as a range of +/−10%), with expressions such as “exactly the center/exact center”, “exactly the middle/exactly middle”, “exactly uniform”, “exactly equal”, “exactly similar”, “completely orthogonal”, “completely parallel”, “completely symmetric”, “completely extend”, “fully axial direction”, “perfectly columnar”, “perfectly cylindrical”, “perfectly ring-shaped”, “perfectly annular”, and “perfectly average” being respectively used as references.

Thus, an expression that does not include the wording such as “substantially” can also include, in concept, a possible expression including the wording such as “substantially”. Conversely, a state expressed using the expression including the wording such as “substantially” may include a state of “exactly/exact”, “completely”, “fully”, or “perfectly”.

In the present disclosure, an expression using “-er than” such as “being larger than A” and “being smaller than A” comprehensively includes, in concept, an expression that includes “being equal to A” and an expression that does not include “being equal to A”. For example, “being larger than A” is not limited to the expression that does not include “being equal to A”, and also includes “being equal to or greater than A”. Further, “being smaller than A” is not limited to “being less than A”, and also includes “being equal to or less than A”.

When the present technology is carried out, it is sufficient if a specific setting or the like is adopted as appropriate from expressions included in “being larger than A” and expressions included in “being smaller than A”, in order to provide the effects described above.

At least two of the features of the present technology described above can also be combined. In other words, the various features described in the respective embodiments may be combined discretionarily regardless of the embodiments. Further, the various effects described above are not limitative but are merely illustrative, and other effects may be provided.

Note that the present technology may also take the following configurations.

(1) An information processing apparatus, including:

an acquisition section that acquires protein information related to a protein;

an input section to which input information that is responsive to an input operation is input, the input operation being performed by a user with respect to the protein information acquired by the acquisition section; and

a generator that generates sequence information related to an amino acid sequence, on the basis of the protein information acquired by the acquisition section and on the basis of the input information input to the input section.

(2) The information processing apparatus according to (1), in which

the generator
generates reflection protein information obtained by the input information being reflected in the protein information, and
predicts the sequence information corresponding to the reflection protein information.
   (3) The information processing apparatus according to (2), in which

the generator predicts the sequence information by performing machine learning using the reflection protein information as input.

(4) The information processing apparatus according to any one of (1) to (3), in which

the protein information includes at least one of a structure of the protein or a function of the protein, and

the input operation includes at least one of an editing operation of editing the structure of the protein, or an editing operation of editing the function of the protein.

(5) The information processing apparatus according to (4), in which

the function of the protein includes at least one of hydrophilicity of the protein or rigidity of the protein.

(6) The information processing apparatus according to any one of (1) to (5), further including

a protein predictor that predicts, as prediction protein information, the protein information corresponding to the sequence information generated by the generator.

(7) The information processing apparatus according to (6), in which

the protein predictor predicts the prediction protein information by performing machine learning using the sequence information as input.

(8) The information processing apparatus according to (6) or (7), in which

the generator corrects the reflection protein information on the basis of a difference between the reflection protein information and the prediction protein information predicted by the protein predictor.

(9) The information processing apparatus according to (2) or (3), further including

a display controller that controls display of a protein image that corresponds to the protein information acquired by the acquisition section.

(10) The information processing apparatus according to (9), in which

the input information includes information that is responsive to the input operation performed with respect to the protein image.

(11) The information processing apparatus according to (9) or (10), in which

the display controller controls display of a reflection protein image that corresponds to the reflection protein information generated by the generator.

(12) The information processing apparatus according to (11), in which

the input information includes information that is responsive to the input operation performed with respect to the reflection protein image.

(13) The information processing apparatus according to any one of (9) to (12), in which

the display controller controls display of a sequence-information image that corresponds to the sequence information predicted by the generator.

(14) The information processing apparatus according to any one of (9) to (13), further including

a protein predictor that predicts, as prediction protein information, the protein information corresponding to the sequence information predicted by the generator, in which

the display controller controls display of a difference image that corresponds to a difference between the reflection protein information and the prediction protein information.

(15) The information processing apparatus according to (14), in which

the difference image includes an image obtained by the reflection protein image and a prediction protein image that corresponds to the prediction protein information overlapping each other.

(16) The information processing apparatus according to (15), in which

the difference image includes an image obtained by the reflection protein image and the prediction protein image overlapping each other, with the difference between the reflection protein information and the prediction protein information being highlighted to be displayed in the included image.

(17) The information processing apparatus according to any one of (9) to (16), further including

a protein predictor that predicts, as prediction protein information, the protein information corresponding to the sequence information generated by the generator, in which

the display controller controls display of at least one of the protein image, a reflection protein image that corresponds to the reflection protein information generated by the generator, or a prediction protein image that corresponds to the prediction protein information such that the at least one of the protein image, the reflection protein image, or the prediction protein image is displayed in at least one of display formats that respectively correspond to a group-of-points image, a polygon image, a mesh image, a surface image, a slice image, and a three-view diagram.

(18) The information processing apparatus according to any one of (1) to (17), in which

the protein information includes template information that corresponds to a template for the protein information.

(19) An information processing method that is performed by a computer system, the information processing method including:

acquiring protein information related to a protein;

inputting input information that is responsive to an input operation that is performed by a user with respect to the acquired protein information; and

generating sequence information related to an amino acid sequence, on the basis of the acquired protein information and on the basis of the input input information.

(20) A program that causes a computer system to perform a process including:

acquiring protein information related to a protein;

inputting input information that is responsive to an input operation that is performed by a user with respect to the acquired protein information; and

generating sequence information related to an amino acid sequence, on the basis of the acquired protein information and on the basis of the input input information.

(21) The information processing apparatus according to any one of (1) to (18), in which

the generator outputs the sequence information in the form of a file.

### REFERENCE SIGNS LIST

1 sequence generation system
4 information processing apparatus
5 protein information
6 sequence information
7 acquisition section
8 input section
9 generator
12 first information processing apparatus
13 second information processing apparatus
19 stereostructure
26 stereostructure generator
27 sequence predictor
28 display controller
29 output section
32 stereostructure image
33 reflection-stereostructure image
34 prediction-stereostructure image
35 difference image
36 reflection stereostructure
37 machine learning model
40 sequence-information image
43 stereostructure predictor
44 stereostructure error calculator
47 correction section

1. An information processing apparatus, comprising:
an acquisition section that acquires protein information related to a protein;
an input section to which input information that is responsive to an input operation is input, the input operation being performed by a user with respect to the protein information acquired by the acquisition section; and
a generator that generates sequence information related to an amino acid sequence, on a basis of the protein information acquired by the acquisition section and on a basis of the input information input to the input section.

2. The information processing apparatus according to claim 1, wherein
the generator
generates reflection protein information obtained by the input information being reflected in the protein information, and
predicts the sequence information corresponding to the reflection protein information.

3. The information processing apparatus according to claim 2, wherein
the generator predicts the sequence information by performing machine learning using the reflection protein information as input.

4. The information processing apparatus according to claim 1, wherein
the protein information includes at least one of a structure of the protein or a function of the protein, and
the input operation includes at least one of an editing operation of editing the structure of the protein, or an editing operation of editing the function of the protein.

5. The information processing apparatus according to claim 4, wherein
the function of the protein includes at least one of hydrophilicity of the protein or rigidity of the protein.

6. The information processing apparatus according to claim 1, further comprising
a protein predictor that predicts, as prediction protein information, the protein information corresponding to the sequence information generated by the generator.

7. The information processing apparatus according to claim 6, wherein
the protein predictor predicts the prediction protein information by performing machine learning using the sequence information as input.

8. The information processing apparatus according to claim 6, wherein
the generator corrects the reflection protein information on a basis of a difference between the reflection protein information and the prediction protein information predicted by the protein predictor.

9. The information processing apparatus according to claim 2, further comprising
a display controller that controls display of a protein image that corresponds to the protein information acquired by the acquisition section.

10. The information processing apparatus according to claim 9, wherein
the input information includes information that is responsive to the input operation performed with respect to the protein image.

11. The information processing apparatus according to claim 9, wherein
the display controller controls display of a reflection protein image that corresponds to the reflection protein information generated by the generator.

12. The information processing apparatus according to claim 11, wherein
the input information includes information that is responsive to the input operation performed with respect to the reflection protein image.

13. The information processing apparatus according to claim 9, wherein
the display controller controls display of a sequence-information image that corresponds to the sequence information predicted by the generator.

14. The information processing apparatus according to claim 9, further comprising
a protein predictor that predicts, as prediction protein information, the protein information corresponding to the sequence information predicted by the generator, wherein
the display controller controls display of a difference image that corresponds to a difference between the reflection protein information and the prediction protein information.

15. The information processing apparatus according to claim 14, wherein
the difference image includes an image obtained by the reflection protein image and a prediction protein image that corresponds to the prediction protein information overlapping each other.

16. The information processing apparatus according to claim 15, wherein
the difference image includes an image obtained by the reflection protein image and the prediction protein image overlapping each other, with the difference between the reflection protein information and the prediction protein information being highlighted to be displayed in the included image.

17. The information processing apparatus according to claim 9, further comprising
a protein predictor that predicts, as prediction protein information, the protein information corresponding to the sequence information generated by the generator, wherein
the display controller controls display of at least one of the protein image, a reflection protein image that corresponds to the reflection protein information generated by the generator, or a prediction protein image that corresponds to the prediction protein information such that the at least one of the protein image, the reflection protein image, or the prediction protein image is displayed in at least one of display formats that respectively correspond to a group-of-points image, a polygon image, a mesh image, a surface image, a slice image, and a three-view diagram.

18. The information processing apparatus according to claim 1, wherein
the protein information includes template information that corresponds to a template for the protein information.

19. An information processing method that is performed by a computer system, the information processing method comprising:
acquiring protein information related to a protein;
inputting input information that is responsive to an input operation that is performed by a user with respect to the acquired protein information; and
generating sequence information related to an amino acid sequence, on a basis of the acquired protein information and on a basis of the input input information.

20. A program that causes a computer system to perform a process comprising:
acquiring protein information related to a protein;
inputting input information that is responsive to an input operation that is performed by a user with respect to the acquired protein information; and
generating sequence information related to an amino acid sequence, on a basis of the acquired protein information and on a basis of the input input information.
